SEQUENCE LISTING (23 sequences)

SEQ ID NO: 1 - DNA, 48 bp, Enterovirus sp.
aattctaatacgactcactatagggagaaggaccggatggccaatcca

SEQ ID NO: 2 - DNA, 24 bp, Enterovirus sp.
cctccggccccggaatgcggctaa

SEQ ID NO: 3 - DNA, 21 bp, Poliovirus sp.
tactttgggtgtccgtgtttc

SEQ ID NO: 4 - DNA, 25 bp, Coxsackievirus A
ataaccccaccccgagtaaacctta

SEQ ID NO: 5 - DNA, 18 bp, Coxsackievirus A
ccgttagcaggcgtggcg

SEQ ID NO: 6 - DNA, 27 bp, Coxsackievirus A
cttcccccgtaactttagaagcttatc

SEQ ID NO: 7 - DNA, 27 bp, Coxsackievirus A
gtatatgctgtacccacggcaaaaaac

SEQ ID NO: 8 - DNA, 23 bp, Coxsackievirus B
cgatcattagcaagcgtggcaca

SEQ ID NO: 9 - DNA, 23 bp, Coxsackievirus B
aacacacaccgatcaacagtcag

SEQ ID NO: 10 - DNA, 23 bp, Coxsackievirus B
ggtcaattactgacgcagcaacc

SEQ ID NO: 11 - DNA, 18 bp, Coxsackievirus B
cccccctccccttaaccg

SEQ ID NO: 12 - DNA, 27 bp, Echovirus 5
ccctcccccgatttgtaacttagaatt

SEQ ID NO: 13 - DNA, 23 bp, Echovirus 9
ccaacggtcaatagacagctcag

SEQ ID NO: 14 - DNA, 25 bp, Echovirus 9
gtttccctttaccccgaatggaact

SEQ ID NO: 15 - DNA, 23 bp, Echovirus 11
caaagctaacccgatcgatagcg

SEQ ID NO: 16 - DNA, 23 bp, Echovirus 12
ataccctcccctcagtaacctag

SEQ ID NO: 17 - DNA, 23 bp, Enterovirus 70
gtacccacggttgaaagcgatga

SEQ ID NO: 18 - DNA, 23 bp, Enterovirus 71
atcaatagtaggcgtaacgcgcc

SEQ ID NO: 19 - DNA, 26 bp, Poliovirus 1
cgcacaaaaccaagttcaaagaaggg

SEQ ID NO: 20 - DNA, 18 bp, Poliovirus 2
cacggagcaggcagtggc

SEQ ID NO: 21 - DNA, 18 bp, Poliovirus 2
cggaagaggcggtcgcga

SEQ ID NO: 22 - DNA, 23 bp, Poliovirus 3
atctcaaccacggagcaggtagt

SEQ ID NO: 23 - DNA, 23 bp, Poliovirus 3
cccccgcaacttagaagcataca

(19) United States
(12) Patent Application Publication
(10) Pub. No.: US 2003/0186222 A1
(43) Pub. Date: Oct. 2, 2003

(54) Rapid detection of enteroviruses in environmental samples by NASBA

(76) Inventor: John H. Paul III, St. Petersburg, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/179,082

(22) Filed: Jun. 25, 2002

Related U.S. Application Data
(60) Provisional application No. 60/301,218, filed on Jun. 27, 2001.

(51) Int. Cl. C12Q 1/70; C12Q 1/68; G06F 19/00; G01N 33/48; G01N 33/50; C12P 19/34

(52) U.S. Cl. 435/5; 435/91.2; 702/20

(57) ABSTRACT

The invention provides an alternate amplification and detection technology for enteroviruses in aquatic samples based on nucleic acid sequence based amplification (NASBA), DNA microarray technology, and electrochemiluminescence. A microscope, in communication with a camera, mounted to an automated stage, in communication with a computer-driven image analysis system is utilized to detect the presence of specific enteroviral RNA exposed to the DNA microarray.

### CROSS-REFERENCE TO A RELATED APPLICATION

This application claims the benefit of U.S. Provisional Application No. 60/301,218, filed Jun. 27, 2001, incorporated herein by reference.

### FIELD OF THE INVENTION

The present invention relates to the use of photolithography systems and methods for detecting specific genetic sequences, more particularly, the use of such systems and methods to detect the presence of enteroviruses in aquatic samples.

### BACKGROUND OF THE INVENTION

Good microbiological water quality in coastal waters is a national priority. With wastewater contaminating such aquatic areas, there is an increased risk of infection. The organisms responsible for infectious risk can include viruses, bacteria, and protozoans. Individuals at risk are those who have increased contact with contaminated water. These individuals nonexclusively include swimmers, divers, and boaters as well as those consuming products harvested from the contaminated water.

Pathogenic organisms such as enteroviruses pose a serious problem to life. Enteroviruses are found worldwide, humans being their only known natural host. The viruses are small, non-enveloped, and isometric, belonging to the family Picornaviridae. The viruses are generally transmitted from person to person by ingestion (for example, fecal-oral route) or from exposure to contaminated water supplies. Furthermore, evidence indicates that the viruses can be spread via the respiratory tract. Once exposed, the virus infects the body via the blood stream and multiplies in the gut mucosa.

Most infections occur during childhood. Although the infections are largely transient, they produce lifelong immunity to the organism. A majority of enteroviral infections result in mild illness; however, enteroviruses can cause an array of different diseases affecting many different organs (for example, neurologic (polio, aseptic meningitis, encephalitis), respiratory (common cold, tonsillitis, pharyngitis, rhinitis) cardiovascular (myocarditis, pericarditis), etc.). The ability to detect the presence of the organisms that cause these conditions is beneficial to the health and welfare of those with increased potential of exposure.

There is no specific treatment for enterovirus infections. In infants, enteroviral meningitis is often confused with bacterial or herpes virus infections resulting in misdiagnosis. Often, children are hospitalized and incorrectly treated with antibiotics and anti-herpes drugs.

While most enteroviral infections are known and documented, these viruses have also been implicated in several chronic diseases, such as juvenile onset of diabetes mellitus, chronic fatigue syndrome, and amyotrophic lateral sclerosis (Lou Gehrig's Disease); however, definitive proof is deficient.

Furthermore, there is a high degree of serological cross reactivity amongst the more than 70 known enteroviruses, which include: Polio 1, 2, 3; Coxsackie A 1-24; Coxsackie B 1-6; ECHO 1-34; Entero 68-71; and Entero 72 (Hepatitis A). To reduce the risk of enteroviral infection, the U.S. EPA proposed legislation mandating the testing of groundwater for the presence of enteroviruses.

The isolation and detection of such organisms is known to be accomplished by reverse transcriptase polymerase chain reaction (RT-PCR) and cell culture. See M. Gilgen et al., (1995) Appl. Environ. Microbiol. 61: 1226-31. Both of these techniques have advantages and disadvantages. While RT-PCR is generally faster, more sensitive, and more specific, it cannot distinguish viable from nonviable viruses. Further, currently available primer sets are not specific among the full suite of human enteroviruses. Currently only about 25 of the more than 70 known enteroviruses can be detected by the RT-PCR method. Thus, use of the RT-PCR assay allows sensitive detection but cannot determine whether the resulting amplicon is from one virus, multiple virus strains, a pathogen, or a vaccine.

DNA microarrays or “genechips” are well known in the art for the study of gene expression. DNA microarrays are orderly arrangements of multiple DNA probes immobilized on a small solid surface. Several techniques are known for fixing probes to solid surfaces and synthesizing probes on surfaces. One such known technique is light activation/fixation. DNA probes, fixed to the surface of the chip, serve as an array to which a target nucleic acid is hybridized. Probes have been developed with the following features: specificity (length and nucleotide content specific for only the target organism); sensitivity (the ability to hybridize efficiently to the target); and stringency (the ability to limit nonspecific hybridizations). Detection of the probe/hybridized target gene or amplified gene segment is typically accomplished with the use of fluorescence. By detection of hybridization at a specific location on the array, the various genes or amplified regions of genes can be identified.

All documents and publications cited herein are incorporated by reference in their entirety, to the extent not inconsistent with the explicit teachings set forth herein.

### BRIEF SUMMARY OF THE INVENTION

The instant invention provides an alternate amplification and detection technology for enteroviruses in aquatic samples based upon nucleic acid sequence based amplification (NASBA). NASBA is an isothermal method of amplifying RNA (Compton, 1991). The process results in an approximate billion-fold amplification of the RNA target in less than two hours and does not utilize Taq polymerase or thermal cycling. It has been described as a self-sustained sequence reaction (3 SR; Guatelli et al., 1990) and transcription-based amplification (TAS; Kwoh et al., 1989).

Key components of NASBA are the conversion of RNA into DNA by the action of reverse transcriptase and the production of RNA by T7 RNA polymerase. First, in the non-cyclic or linear phase of the reaction, a primer (P1) is bound at the 3′ end of messenger RNA (mRNA). This primer is unusual in that it contains a T7 RNA polymerase promoter. Second, AMV reverse transcriptase converts the molecule to a RNA/DNA hybrid. RNAse H specifically degrades the RNA in the hybrid and the AMV reverse transcriptase converts the single stranded DNA into double stranded DNA. Finally, T7 RNA polymerase recognizes the T7 RNA polymerase promoter, initiating the cyclic phase. Antisense RNA product is produced and the AMV reverse transcript makes a DNA/RNA hybrid. RNAse H degrades the RNA, a duplex DNA molecule is synthesized, T7 RNA polymerase makes RNA, and the cycle continues.

The method described below combines current sample concentration and NASBA technology with novel nucleotide primers to amplify the viral RNA. Aquatic samples are obtained and can be concentrated by any method known in the art (for example, charged filters, filterite cartridges, vortex flow filtration, etc.) or, alternatively, left unconcentrated. The viral RNA is extracted by a combination of heating and Rneasy extraction. Utilizing novel enteroviral primers, the RNA is amplified using NASBA technology. The RNA is then detected using a method known in the art (for example, by gel electrophoresis, molecular probing, or electrochemiluminescence (ECL)). When using ECL, detection probes specific to virus type (such as poliovirus, Coxsackievirus, echovirus, etc.) are utilized.

In a further embodiment, an assay and method are provided for detecting an organism such as a virus, more particularly; and in a preferred embodiment, an enterovirus. This process comprises the steps of fixing an oligonucleotide probe to an organic spacer at an end, the spacer in turn being connected to a linker that is adapted to be photolinked to the coated surface of the microarray. The affixing light source is reflected onto microspots located on the surface of the microarray by means of a spatial light modulator.

Another embodiment of the present invention includes a microarray system for detecting specific compositional sequences, such as, but not limited to, oligonucleotide sequences specific to known enteroviruses. Such a system is used in detecting pathogenic viruses in clinical or environmental settings, and can be used in the field as an indicator of pollution levels and other conditions dangerous for human and other life.

### BRIEF DESCRIPTION OF THE DRAWINGS

FIG. 1 is a schematic diagram of a system for performing viral detection.

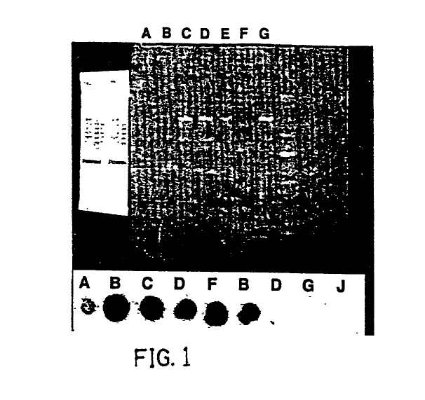

FIG. 2 is a gel electrophoresis illustrating the effects of NASBA amplification of enteroviral genomes.

### BRIEF DESCRIPTION OF THE SEQUENCES

SEQ ID NO: 1 is the nucleotide sequence for primer Ent P1 (=JP127).

SEQ ID NO: 2 is the nucleotide sequence for primer Ent P2 (=JP128).

SEQ ID NO: 3 is the nucleotide sequence for a probe specific for detecting poliovirus.

SEQ ID NO: 4 is the nucleotide sequence for a probe specific for detecting Coxsackievirus A9.

SEQ ID NO: 5 is the nucleotide sequence for a probe specific for detecting Coxsackievirus A16.

SEQ ID NO: 6 is the nucleotide sequence for a probe specific for detecting Coxsackievirus A21.

SEQ ID NO: 7 is the nucleotide sequence for a probe specific for detecting Coxsackievirus A24.

SEQ ID NO: 8 is the nucleotide sequence for a probe specific for detecting Coxsackievirus B1.

SEQ ID NO: 9 is the nucleotide sequence for a probe specific for detecting Coxsackievirus B3.

SEQ ID NO: 10 is the nucleotide sequence for a probe specific for detecting Coxsackievirus B4.

SEQ ID NO: 11 is the nucleotide sequence for a probe specific for detecting Coxsackievirus B5.

SEQ ID NO: 12 is the nucleotide sequence for a probe specific for detecting Echovirus 5.

SEQ ID NO: 13 is the nucleotide sequence for a probe specific for detecting Echovirus 9 (ECHOV9XX).

SEQ ID NO: 14 is the nucleotide sequence for a probe specific for detecting Echovirus 9 (EV9GENOME).

SEQ ID NO: 15 is the nucleotide sequence for a probe specific for detecting Echovirus 11.

SEQ ID NO: 16 is the nucleotide sequence for a probe specific for detecting Echovirus 12.

SEQ ID NO: 17 is the nucleotide sequence for a probe specific for detecting Enterovirus 70.

SEQ ID NO: 18 is the nucleotide sequence for a probe specific for detecting Enterovirus 71.

SEQ ID NO: 19 is the nucleotide sequence for a probe specific for detecting Poliovirus 1.

SEQ ID NO: 20 is the nucleotide sequence for a probe specific for detecting Poliovirus 2 (POL2CG1).

SEQ ID NO: 21 is the nucleotide sequence for a probe specific for detecting Poliovirus 2 (PIPOLS2).

SEQ ID NO: 22 is the nucleotide sequence for a probe specific for detecting Poliovirus 3 PIPO3XX).

SEQ ID NO: 23 is the nucleotide sequence for a probe specific for detecting Poliovirus 3.

### DETAILED DISCLOSURE OF THE INVENTION

An exemplary embodiment comprises a system for detecting an RNA virus. This aspect of the invention comprises a series of biochemical steps preparatory to commencing the detection assay.

Aquatic samples are obtained and are tested, either unconcentrated or after concentration by a method known in the art (for example, charged filters, filterite cartridges, vortex flow filtration, etc.). Current sample concentration and NASBA technology is combined with novel nucleotide primers to amplify the viral RNA contained therein. The viral RNA is extracted by a combination of heating and Rneasy extraction. Utilizing novel enteroviral primers, the RNA is amplified using NASBA technology. The RNA sample is exposed to a microarray containing probes specific to enterovirus type. Utilizing electrochemiluminescence (ECL), the specificity of the RNA is then detected using a microscope-camera-computer combination that detects the reaction occurring between the viral RNA obtained from the aquatic sample and the probes contained on the microarray.

Following are examples illustrating procedures for practicing the invention. These examples should be construed to include obvious variations and not construed as limiting. Unless noted otherwise, all solvent mixture proportions are by volume and all percentages are by weight.

### EXAMPLE 1

First, an oligonucleotide probe for a desired specific RNA virus is designed, for example, a human pathogenic enterovirus. In a particular embodiment, a 600-base-pair segment of an enteroviral 5′untranslated region (5′UTR) is used, from which oligonucleotide probes are selected. Exemplary viruses include the polioviruses, Coxsackie A and B viruses, echoviruses, and other enteroviruses. Preferably all probes are designed to have melting temperatures (Tₘ) within a predetermined range, for example, about 1° C. of each other. This criterion permits the development of a multiprobe microarray with stringency wash conditions that limit nonspecific hybridizations. Selected oligonucleotides are compared to determine specificity to target and nontarget organisms. Once determined a single specific probe for each virus is then selected.

Next, the probes are tested in hybridization assays with known enteroviral standards. These standards can be obtained from a gene bank. The specificity of the probes is determined by hybridization to target and nontarget nucleic acids in standard membrane hybridization assays.

From the selected enteroviral isolates are amplified a 600-base-pair portion of the 5′UTR using RT-PCR. The sensitivity of the assay is addressed by using an attenuated enterovirus stock of known concentration, a dilution series, and dot blot hybridization to an existing gene probe to determine the limits of detection. The process is repeated using a fluorescently labeled nucleotide to determine its effect on efficient amplification.

A 9:1 mixture of nonlabeled dCTP and fluorescently labeled dCTP does not inhibit the reaction, and that labeled amplicon can be seen with the human eye when using gel electrophoresis, without staining, and a UV-transilluminator. To optimize amplicon signal strength, various ratios of the nonlabeled versus fluorescently labeled dCTP are evaluated.

To address the specificity of each probe and to define optimal stringency conditions, large-scale arrays are created using a dot blot format, charged nylon filter paper, biotinylated probes and light (for linking probes to the filter). Replicate nylon filters are made for each probe and each nylon array contains fixed copies of every probe in a predetermined order. Amplicons are obtained from each enterovirus isolate by RT-PCR or NASBA. Individual amplicons are hybridized to the filters and the specificity of each probe visualized using electrochemiluminescence. Mixtures of stock viruses are also used to evaluate simultaneous detection of multiple amplicons. An evaluation is made of conditions for blocking (limiting nonspecific hybridizations and binding to the filter surface), stringency (limiting nonspecific hybridizations of sequences that are close to complementary), and hybridization (hybridization time and reagent types) in order to optimize the detection assay.

Using the microarray technology disclosed above, the designed oligonucleotides are affixed to a surface in an array pattern. A digital mirror device, such as those offered by Texas Instruments, comprises a processor-controlled array of miniature mirrors. Each mirror in the device can be oriented so that light impinging thereon can be focused on a desired point.

The system has a light source, for example, a laser emitting light that passes through a series of optical elements and impinges upon the digital mirror device. Under processor control, the light is then selectively focused onto a substrate, for example, a chip exposed to at least one probe. An exemplary spot size of the light focused by the mirror device is about 30 μm, although different sizes may be utilized depending on the need, as would be readily apparent to the skilled artisan.

### EXAMPLE 2

The method of creating the array chip of the present invention comprises the steps of providing a linker, such as a commercially available reagent, having two reactive groups, on each molecule. One reactive group is adapted to be chemically linked to a biological molecule.

A synthesized oligonucleotide probe is labeled at the 5′ end with a spacer and an amine group that is adapted to react with the linker (for example, sulfosuccinimidyl(perfluoroazidogenzamido) ethyl-1,3 dithiopropionate). Each probe comprises a spacer, in an exemplary embodiment a 24-carbon spacer between the probe's 5′ end and the amine group. The spacer is for limiting steric hindrance upon the probes being attached to the array. This technique is used since it has been found that if the probe is attached directly to a solid surface, the entire sequence thereof may not be available for target hybridization, which could limit specificity and sensitivity.

The linker is photolinked to the surface of a microarray coated with 3-aminopropyltriethozysilane. The affixing light source is reflected onto microspots on the array by a spatial light modulator. The oligonucleotides are spatially arranged one oligonucleotide at a time to the microarray. The resulting orientation is: linker-label-spacer-5′probe-3′.

After the oligonucleotide probe is attached to the linker, the probe/linker combination is attached to a solid surface via the linker's second reactive group. This second group can comprise a photoactivated cross-linking agent. Thus the composition now comprises an array surface—linker label—spacer—5′-probe-3′. Experimental data resulting from the testing of the linking of the probe to a glass substrate using a photoreactive linker indicate that each UV-fixed linker/probe site contains a probe.

Using the created microarray and fluorescent labeling of the RT-PCR or NASBA amplicons, optimal hybridization conditions such as prehybridization, hybridization temperature, and the salt concentration in the hybridization solution, identified using the dot blot/nylon membrane format, can be defined and confirmed. Different blocking reagents can be analyzed to limit or eliminate the potential for any background signals produced by nonspecific binding of amplicon to the surface of the chip.

Further, the specificity and sensitivity of each attached array probe can be evaluated using fluorescently labeled amplicon(s) obtained from each desired virus. As with the dot blot format, single and mixed amplicons can be used to define conditions as outlined.

Preferably, labeling and probe attachment steps are performed in a light-shielded environment to avoid the potential for premature or unwanted photoactivation of the linker.

### EXAMPLE 3

In a preferred embodiment NASBA technology, in combination with a concentration of the virus, is utilized. This step comprises filtering a desired volume of water (typically approximately 110 liters) by a method such as is known in the art (Filterite filter DFN 0.45-10UN; Filterite/MEMTEC A. Corp., Timonium, Md.; Standard Methods for the Examination of Water and Wastewater, 20^{th} ed., American Public Health Assoc., Washington, D.C., 1998). Viruses are eluted with beef extract (pH 9.5) and concentrated using organic flocculation. As an alternative, the water canbe filtered using vortex flow filtration. See J. H. Paul et al., (1991) Concentration of viruses and dissolved DNA from aquatic environments by vortex flow filtration Appl. Environ. Microbiol 57: 2197-204. The viral concentrate or standard poliovirus, for example, is stored at −20° C. until extraction.

Extraction is accomplished by taking a desired amount of the concentrate or enteroviral standard (for example, poliovirus), diluted to 100 μl in DEPC D1 (diethylpyrocarbonate-treated deionized water). An exemplary kit for accomplishing extraction comprises the Rneasy kit (Qiagen, Santa Clarita, Calif.). Dilutions of the enterovirus are made to concentrations of 9×10⁶, 9×10⁴, 9×10³, and 9×10² enteroviruses in a 1.5-ml microfuge tube. Then 350 μl of RLT buffer, containing 10 μl β-mercaptoethanol per 1.0 ml RLT buffer, is added to the tubes, and the tubes are capped and placed in a 95° C. water bath for 10 minutes, followed by placement in an ice bath for 5 minutes. An amount of ethanol, here 250 μl, is added and mixed well by pipetting. This mixture (typically 700 μl), including any precipitate, is added to the spun column, and the tube is placed in a 2-ml collection tube, which is placed in a microfuge for 15 seconds at ≧10,000 rpm. 700 μl of buffer RW1 is pipetted into the column and microfuged for 15 seconds at ≧10,000 rpm to wash. Then 500 μl of RPE buffer is pipetted into the tube, using a new collection 2.0-ml tube, and microfuged for 15 seconds at ≧10,000 rpm. 500 μl RPE is pipetted onto the column and centrifuged for 2 minutes at maximum speed to dry the column using the same collection tube. The mixture is transferred to a new 1.5-ml collection tube, with 30 μl Rnase-free water pipetted, and the tube is spun for 1 minute to elute. Then 1 U (unit) of Rnasin (Promega) per microliter of sample is added.

Amplification of the enterovirus (here, for example, poliovirus) is preferably accomplished with the use of a kit (Organon Teknika (Durham, N.C.)), using two primer sequences, EntP1 (=JP127) and EntP2 (=JP128). ENTP1 comprises 5′-AAT-TCT-AAT-ACG-ACT-CAC-TAT-AGG-GAG-AAG-GAC-CGG-ATG-GCC-AAT-CCA-A-3′ (SEQ ID NO: 1); EntP2 comprises 5′-CCT-CCG-GCC-CCT-GAA-TGC-GGC-TAA-3′ (SEQ ID NO: 2). The primers are gel purified. Lyophilized primers are taken up in sterile DEPC-D1 to a final concentration of 100 μM, and aliquotted. The aliquotted samples can be utilized immediately by diluting to 10 μM, or they can be frozen for future use. If frozen prior to use, they are thawed and diluted to 10 μM.

A clean workbench should preferably be set up with UV-sterilization and hot blocks set at 65 and 41° C. Using a kit such as the Organon Teknika kit, add 50 μl accusphere diluent to lyophilized accusphere, and vortex well. Then 5 μl of each diluted (10 μM) EntP1 and EntP2 primers are added to 50 μl dissolved accusphere for a total of 60 μl, which is sufficient for 11-12 reactions.

70 mM KCl is prepared. For example, 70 mM KCl can be prepared utilizing the NASBA kit by adding 8.4 μl NASBA KCl and 51.6 μl NASBA water. The 60 μl primer/accusphere mix is combined with the 60 μl KCl. For a positive control, the control contained in the kit can be used. In this case, 50 μl NASBA water plus the lyophilized NASBA control are added.

The reaction is set up by adding 5 μl D1+10 μl of primer/KCl mixture to a sterile 1.5-ml microfuge tube, which serves as a blank. For a poliovirus or unknown sample, 5 μl D1+10 μl primer/KCl mixture is added to a sterile 1.5 ml microfuge tube. For a kit control, aliquot out 15 μl of the positive control mixture to a sterile 1.5 ml microfuge tube.

The tubes are placed in the 65° C. hot block for 5 minutes and in a 41° C. hot block for 5 minutes, and 5 μl of the NASBA enzyme mixture is added, with mixing accomplished preferably by flicking, not trituration. The tubes are incubated in the 41° C. hot block for 5 minutes, then pulse spun in a microfuge for 1-2 sec, incubated for 90 minutes in the 41° C. hot block, removed from the hot block, and immediately utilized for detection. Alternatively, if the samples are not used immediately, they can be frozen at −80° C. immediately and stored.

It should be noted that utmost care should be taken to keep extraction areas separate from amplification areas. At all times, positive controls should be kept separated from other samples. For example, the positive controls should never be in the same rack as negatives or unknowns. To further decrease the chance of cross-contamination, aerosol pipette tips should be used, gloves worn, and gloves changed frequently.

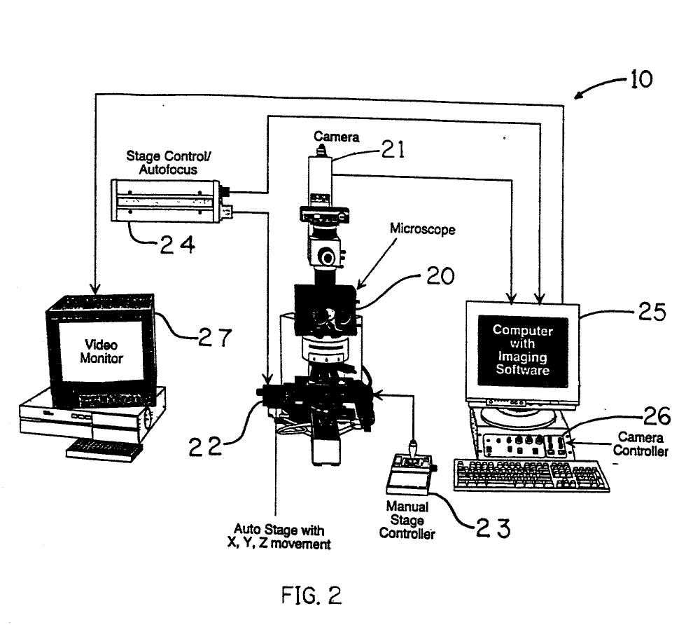

Detection is accomplished using, for example, a 7% acrylamide gel, run for 3-5 h, followed by ethidium bromide staining (FIG. 2). Alternatively, dot blotting and probing can be used. A list of preferred probes is provided in Table 1 below. One probe useful for poliovirus comprises 5′-TAC-TTT-GGG-TGT-CCG-TGT-TTC-3′(SEQ ID NO: 3). The probes can be labeled, such as by using a Tropix, Inc. (Bedford, Mass.), Southern Star Chemiluminesent Detection System, version A.2. Detection of specific enteroviral types can be accomplished by probing with specific viral-type oligonucleotide probes. Alternatively, ECL probes can be designed for specific viral types.

TABLE 1

| Enterovirus | Probe Sequence | SEQ ID NO: |
|---|---|---|
| Poliovirus | TACTTTGGGTGTCCGTGTTTC | 3 |
| Coxsackie | ATAACCCCACCCCGAGTAAACCTTA | 4 |
| A9 | | |
| Coxsackie | CCGTTAGCAGGCGTGGCG | 5 |
| A 16 | | |
| Coxsackie | CTTCCCCCGTAACTTTAGAAGCTTATC | 6 |
| A 21 | | |
| Coxsackie | GTATATGCTGTACCCACGGCAAAAAAC | 7 |
| A 24 | | |
| Coxsackie | CGATCATTAGCAAGCGTGGCACA | 8 |
| B1 | | |
| Coxsackie | AACACACACCGATCAACAGTCAG | 9 |
| B3 | | |
| Coxsackie | GGTCAATTACTGACGCAGCAACC | 10 |
| B4 | | |
| Coxsackie | CCCCCCTCCCCTTAACCG | 11 |
| B5 | | |
| Echo 5 | CCCTCCCCCGATTTGTAACTTAGAATT | 12 |
| Echo 9 | CCAACGGTCAATAGACAGCTCAG | 13 |
| (ECHOV9XX) | | |
| Echo 9 | GTTTCCCTTTACCCCGAATGGAACT | 14 |
| (EV9GENOME) | | |
| Echo 11 | CAAAGCTAACCCGATCGATAGCG | 15 |
| Echo 12 | ATACCCTCCCCTCAGTAACCTAG | 16 |
| Entero 70 | GTACCCACGGTTGAAAGCGATGA | 17 |
| Entero 71 | ATCAATAGTAGGCGTAACGCGCC | 18 |
| Polio 1 | CGCACAAAACCAAGTTCAAAGAAGGG | 19 |
| Polio 2 | CACGGAGCAGGCAGTGGC | 20 |
| (POL2CG1) | | |
| Polio 2 | CGGAAGAGGCGGTCGCGA | 21 |
| (PIPOLS2) | | |
| Polio 3 | ATCTCAACCACGGAGCAGGTAGT | 22 |
| (PIPO3XX) | | |
| Polio 3 | CCCCCGCAACTTAGAAGCATACA | 23 |

Electrochemiluminescence detection is a preferred method for detecting NASBA amplified RNA targets. Two detection oligonucleotides are employed in solution hybridization. The first is bound to a magnetic bead, serving to bind to the target and immobilize the amplicon to a magnetic electrode. The second is bound to ruthenium and is complimentary to the second part of the amplicon. As a result, two specific target hybridizations are required to verify the presence of the amplicon. A charge is applied across the electrode and the ruthenium radical gives off light that is detected by the microscope-camera-computer combination described below.

Referring now to FIG. 2, the detection of positive hybridization can be accomplished by means of computer operated epifluorescence microscope system 10. The microscope 20, for example, the Olympus BX60 or GSI Luminics ScanArray 5000 microarray reader, is in communication with a camera 21 (for example, MTI V# 1000 Silicon Intensified Target camera) and mounted to an automated stage 22, (for example, Ludl Electronics Products MAC 2000, drivable in three axes (for example, x, y, and auto-focus z)). The automated stage 22 is in communication with both a manual stage control 23 and a computer controlled stage control and auto-focus 24. The manual stage control 23 allows for the manual control of the automated stage 22. The computer controlled stage control and auto-focus 24 is in further communication with a computer driven image analysis system 25 for computer controlled operation of the automated stage 22. The camera 21 is in electronic communication with the computer-driven image analysis system 25, having a camera controller 26 and software (not shown) that can establish and follow an automated tracking pattern for the automated stage 22, as well as capture images utilizing frame-grabber (not shown) or similar technology (for example, Image Pro Plus (Media Cybernetics)). A video monitor 27 can be added in communication with the camera 21 or computer driven image analysis system 25 for viewing purposes. It is possible to use lower power (for example, 40×-200×), depending upon the spacing of array dots produced by the digital mirror array device. As an example, a 200× objective enables an area of about 0.78 mm² to be viewed at one time. The computer operated epifluorescence microscope system 10 detects the chemiluminesent reaction occurring between the probe and the target enterovirus amplicon, thereby indicating the presence of such enterovirus.

Inasmuch as the preceding disclosure presents the best mode devised by the inventor for practicing the invention and is limited to enable one skilled in the pertinent art to carry it out, it is apparent that methods incorporating modifications and variations will be obvious to those skilled in the art. As such, it should not be construed to be limited thereby but should include such aforementioned obvious variations and be limited only by the spirit and scope of the following claims.

1. A method of detecting the presence of at least one specific enterovirus in an aquatic sample comprising:
obtaining an aquatic sample;
obtaining a DNA microarray, said DNA microarray having at least one enterovirus specific oligonucleotide probe affixed thereto, said enterovirus specific oligonucleotide probe having a label attached thereto;
amplifying enteroviral RNA contained in said aquatic sample by utilizing at least one purified primer;
exposing said enteroviral RNA to said DNA microarray; and
detecting a reaction occurring between said enteroviral RNA and said labeled enterovirus specific oligonucleotide probe, thereby confirming the presence of the enterovirus to which said enterovirus specific oligonucleotide probe is specific.

2. The method of claim 1 wherein said detecting step comprises using an epifluorescent microscope in communication with a camera mounted to an automated stage in communication with a computer-driven image analysis system.

3. The method of claim 2 wherein said computer-driven analysis system further comprises tracking pattern software to operate said automated stage.

4. The method of claim 3 wherein said computer-driven analysis system further comprises image capture technology.

5. The method of claim 2 wherein said automated stage is drivable in three axes.

6. The method of claim 1 wherein said amplifying step utilizes nucleic acid based sequence amplification.

7. The method of claim 1 wherein said primer is SEQ ID NO: 1.

8. The method of claim 1 wherein said primer is SEQ ID NO: 2.

9. The method of claim 1 wherein said primer is gel purified.

10. The method of claim 1 wherein said amplifying step utilizes reverse transcriptase polymerase chain reaction.

11. The method of claim 1 wherein said enteroviral specific oligonucleotide probe is selected from a 600 base pair segment of enteroviral 5′untranslated region (5′UTR).

12. The method of claim 1 further comprising the step of exposing said DNA microarray to at least one known enteroviral standard.

13. The method of claim 1 wherein said at least one enterovirus specific oligonucleotide probe is selected from the group consisting of SEQ ID NOS: 3, 4, 5, 6, 7, 8, 9, 10, 11, 12, 13, 14, 15, 16, 17, 18, 19, 20, 21, 22, and 23.

14. The method of claim 12 wherein said known enteroviral standard is selected from the group consisting of: Polio 1, 2, 3; Coxsackie A 1, 2, 3, 4, 5, 6, 7, 8, 9, 10, 11, 12, 13, 14, 15, 16, 17, 18, 19, 20, 21, 22, 23, 24; Coxsackie B 1, 2, 3, 4, 5, 6; ECHO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34; Entero 68, 69, 70, 71; and, Entero 72 (Hepatitis A).

15. The method of claim 1 wherein said enterovirus is selected from the group consisting of: Polio 1, 2, 3; Coxsackie A 1, 2, 3, 4, 5, 6, 7, 8, 9, 10, 11, 12, 13, 14, 15, 16, 17, 18, 19, 20, 21, 22, 23, 24; Coxsackie B 1, 2, 3, 4, 5, 6; ECHO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34; Entero 68, 69, 70, 71; and, Entero 72 (Hepatitis A).

16. The method of claim 1 wherein said labeled enterovirus specific oligonucleotide probe comprises an organic spacer between said label and said enterovirus specific oligonucleotide probe.

17. The method of claim 16 wherein said organic spacer is attached to the 5′ end of said enterovirus specific oligonucleotide probe.

18. The method of claim 18 wherein said organic spacer is a 24-carbon spacer.

19. The method of claim 1 further comprising locating a linker between said label and a surface of said DNA microarray.

20. The method of claim 19 wherein said linker is sulfosuccinimidyl (perfluoroazidogenzamido)ethyl- 1,3 dithioproponate.

21. The method of claim 20 wherein the surface of said DNA microarray has been coated with 3-aminopropyltriethoxysilane prior to affixing said enterovirus specific oligonucleotide probe to said DNA microarray.

22. The method of claim 19 wherein said linker is photolinked to a surface of said DNA microarray.

23. The method of claim 1 wherein said label is fluorescent.

24. The method of claim 1 wherein said label is chemiluminesent.

25. The method of claim 1 wherein said DNA microarray further comprises multiple enterovirus specific oligonucleotide probes.

26. The method of claim 25 wherein said multiple enterovirus specific oligonucleotide probes have a melting temperature within a predetermined range of each other.

27. The method of claim 26 wherein said predetermined range is about 1° C.

28. An apparatus for detecting enteroviral presence in aquatic samples comprising:
a means to amplify viral RNA utilizing at least one purified primer;
a DNA microarray to which said viral RNA is exposed, said DNA microarray having at least one enterovirus specific oligonucleotide probe affixed thereto, said enterovirus specific oligonucleotide probe having a label attached thereto; and
detection means for detecting a reaction occurring between said enteroviral RNA and said labeled enterovirus specific oligonucleotide probe, thereby confirming the presence of the enterovirus to which said enterovirus specific oligonucleotide probe is specific.

29. The apparatus of claim 28 wherein said detection means comprises an epifluorescent microscope in communication with a camera mounted to an automated stage in communication with a computer-driven image analysis system.

30. The apparatus of claim 28 wherein said computer-driven analysis system further comprises tracking pattern software to operate said automated stage.

31. The apparatus of claim 28 wherein said computer-driven analysis system further comprises image capture technology.

32. The apparatus of claim 28 wherein said automated stage is drivable in three axes.

33. The apparatus of claim 28 wherein said amplification means is nucleic acid based sequence amplification.

34. The apparatus of claim 28 wherein said purified primer is SEQ ID NO: 1.

35. The apparatus of claim 28 wherein said purified primer is SEQ ID NO: 2.

36. The apparatus of claim 28 wherein said purified primer is gel purified.

37. The apparatus of claim 28 wherein said amplification means is reverse transcriptase polymerase chain reaction.

38. The apparatus of claim 28 wherein said enteroviral specific oligonucleotide probe is selected from a 600 base pair segment of enteroviral 5′ untranslated region (5′ UTR).

39. The apparatus of claim 28 wherein said enterovirus specific oligonucleotide probe is exposed to at least one known enteroviral standard.

40. The apparatus of claim 28 wherein said enterovirus specific oligonucleotide probe is selected from the group consisting of SEQ ID NOS: 3, 4, 5, 6, 7, 8, 9, 10, 11, 12, 13, 14, 15, 16, 17, 18, 19, 20, 21, 22, and 23.

41. The apparatus of claim 39 wherein said known enteroviral standard is selected from the group consisting of: Polio 1, 2, 3; Coxsackie A 1, 2, 3,4, 5, 6, 7, 8, 9, 10, 11, 12, 13, 14, 15, 16, 17, 18, 19, 20, 21, 22, 23, 24; Coxsackie B 1, 2, 3, 4, 5, 6; ECHO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34; Entero 68, 69, 70, 71; and, Entero 72 (Hepatitis A).

42. The apparatus of claim 28 wherein said enterovirus is selected from the group consisting of: Polio 1, 2, 3; Coxsackie A 1, 2, 3, 4, 5, 6, 7, 8, 9, 10, 11, 12, 13, 14, 15, 16, 17, 18, 19, 20, 21, 22, 23, 24; Coxsackie B 1, 2, 3, 4, 5, 6; ECHO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34; Entero 68, 69, 70, 71; and, Entero 72 (Hepatitis A).

43. The apparatus of claim 28, further comprising an organic spacer located between said label and said enterovirus specific oligonucleotide probe.

44. The apparatus of claim 43, wherein said organic spacer is attached to the 5′ end of said enterovirus specific oligonucleotide probe.

45. The apparatus of claim 44 wherein said organic spacer is a 24-carbon spacer.

46. The apparatus of claim 28, further comprising a linker located between said spacer and a surface of said DNA microarray.

47. The apparatus of claim 46 wherein said linker is sulfosuccinimidyl (perfluoroazidogenzamido)ethyl-1,3 dithioproponate.

48. The apparatus of claim 47 wherein the surface of said DNA microarray has been coated with 3-aminopropyltriethoxysilane prior to affixing said enterovirus specific oligonucleotide probe to said DNA microarray.

49. The apparatus of claim 48 wherein said linker is photolinked to a surface of said DNA microarray

50. The apparatus of claim 28 wherein said label is fluorescent.

51. The apparatus of claim 28 wherein said label is chemiluminesent.

52. The apparatus of claim 28 wherein said DNA microarray further comprises multiple enterovirus specific oligonucleotide probes.

53. The apparatus of claim 52 wherein said multiple enterovirus specific oligonucleotide probes have a melting temperature within a predetermined range.

54. The apparatus of claim 53 wherein said predetermined range is about 1° C.

55. A DNA microarray having at least one enterovirus specific oligonucleotide probe affixed thereto, said enterovirus specific oligonucleotide probe having an organic spacer attached thereto at a first end of said organic spacer, said spacer having a label attached at a first end of said label to a second end of said spacer, said label affixed at a second end of said label to a first end of a linker, said linker attached at a second end to a surface of said DNA microarray.

56. The DNA microarray of claim 55, wherein said at least one enterovirus specific oligonucleotide probe is selected from the group consisting of: SEQ ID NOS: 3, 4, 5, 6, 7, 8, 9, 10, 11, 12, 13, 14, 15, 16, 17, 18, 19, 20, 21, 22, and 23.

57. The DNA microarray of claim 56 further comprising multiple enterovirus specific oligonucleotide probes.

58. The DNA microarray of claim 57 wherein said multiple probes have a melting temperature within a predetermined range.

59. The DNA microarray of claim 58 where said predetermined range is about 1° C.

60. The DNA microarray of claim 55 wherein said label is fluorescent.

61. The DNA microarray of claim 55 wherein said label is chemiluminesent.

62. The DNA microarray of claim 55 wherein said organic spacer is a 24 carbon spacer.

63. The DNA microarray of claim 55 wherein said linker is sulfosuccinimidyl (perfluoroazidogenzamido)ethyl-1,3 dithioproponate.

64. The DNA microarray of claim 63 further comprising a chemical preparation coated on the surface of said DNA microarray prior to affixing said enterovirus specific oligonucleotide probe.

65. The DNA microarray of claim 64 wherein said chemical preparation comprises 3-aminopropyltriethoxysilane.

66. The DNA microarray of claim 55 wherein said linker is photolinked to said surface of said DNA microarray.
